(19) United States
(12) Patent Application Publication
(10) Pub. No.: US 2024/0100352 A1
(43) Pub. Date: Mar. 28, 2024

(54) Phototherapy device

(71) Applicant: Coretronic Corporation, Hsin-Chu (TW)

(72) Inventors: CHUNG-JEN OU, Hsin-Chu (TW); YU-MIN CHEN, Hsin-Chu (TW); MING-WEI TSAI, Hsin-Chu (TW); CHIEN-CHIH CHEN, Hsin-Chu (TW)

(21) Appl. No.: 18/369,855

(22) Filed: Sep. 19, 2023

(30) Foreign Application Priority Data
Sep. 28, 2022 (CN) 202211190092.5

(51) Int. Cl. A61N 5/06

(52) U.S. Cl. CPC A61N 5/0616; A61N 2005/0626; A61N 2005/0652; A61N 2005/0663

(57) ABSTRACT

A phototherapy device includes a base, at least one light conversion device and a light source module. The base has an installation slot. The light conversion device is detachably arranged in the installation slot. Each light conversion device includes a plurality of light conversion patterns. The light source module is arranged on a side of the base and configured to provide an excitation beam to the light conversion patterns, so that each of the light conversion patterns emits a converted beam. In this way, the light conversion device of the phototherapy device can be replaced according to the user's needs.

### CROSS-REFERENCE TO RELATED APPLICATION

This application claims the priority benefit of China application (No. 202211190092.5), filed on Sep. 28, 2022. The entirety of the above-mentioned patent application is hereby incorporated by reference herein and made a part of this specification.

### FIELD OF THE INVENTION

The invention relates to a phototherapy device, and more particularly to a phototherapy device that performs phototherapy through the light generated by a color conversion material.

### BACKGROUND OF THE INVENTION

It is known that different wavelengths of light can have different effects on the human body at present. Part of the wavelength of light can promote wound healing, part of the wavelength of light can alleviate skin sensitivity symptoms, and part of the wavelength of light can stimulate muscle repair. This feature is used by people, and a medical treatment called phototherapy was born.

At present, there are many technologies using light emitting diodes (LEDs) or lasers for phototherapy. However, the wavelength of the light emitted by the current phototherapy device is relatively fixed, and there are few customized designs to meet different needs. Thus, the users have to change a phototherapy device if they need to use light of other wavelengths in the phototherapy treatments, which is very inconvenient.

The information disclosed in this “BACKGROUND OF THE INVENTION” section is only for enhancement understanding of the background of the invention and therefore it may contain information that does not form the prior art that is already known to a person of ordinary skill in the art. Furthermore, the information disclosed in this “BACKGROUND OF THE INVENTION” section does not mean that one or more problems to be solved by one or more embodiments of the invention were acknowledged by a person of ordinary skill in the art.

### SUMMARY OF THE INVENTION

The invention provides a phototherapy device, and the user can replace the color conversion device on the phototherapy device according to the demand.

Other advantages and objectives of the invention may be further illustrated by the technical features broadly embodied and described as follows.

In order to achieve one or a portion of or all of the objectives or other objectives, the phototherapy device provided by the present invention includes a base, at least one light conversion device and a light source module. The base has an installation slot. The light conversion device is detachably arranged in the installation slot. Each light conversion device includes a plurality of light conversion patterns. The light source module is arranged on a side of the base and configured to provide an excitation beam to the light conversion patterns, so that each of the light conversion patterns emits a converted beam.

In the phototherapy device of each embodiment of the present invention, because the base has an installation slot allowing the light conversion device to have a detachable arrangement, the user can select or replace a specific light conversion device to convert the excitation beam emitted by the light source module into a beam with a specific wavelength and phototherapy pattern required for phototherapy according to different treatments or needs in a single phototherapy device.

Other objectives, features and advantages of the invention will be further understood from the further technological features disclosed by the embodiments of the invention wherein there are shown and described preferred embodiments of this invention, simply by way of illustration of modes best suited to carry out the invention.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1 is a schematic view of a phototherapy device according to an embodiment of the present invention;

FIG. 2 is a schematic view of a light conversion device in FIG. 1;

FIG. 3 is a schematic view of a light source module, a light conversion device and a light spot projected on a reference surface of a phototherapy device according to an embodiment of the present invention;

FIG. 4 is a schematic view of another light conversion device in the embodiment of FIG. 3; and

FIG. 5 is a block diagram of a phototherapy device according to an embodiment of the present invention.

### DETAILED DESCRIPTION OF PREFERRED EMBODIMENTS

In the following detailed description of the preferred embodiments, reference is made to the accompanying drawings which form a part hereof, and in which is shown by way of illustration specific embodiments in which the invention may be practiced. In this regard, directional terminology, such as “top”, “bottom”, “front”, “back”, etc., is used with reference to the orientation of the Figure(s) being described. The components of the invention can be positioned in a number of different orientations. As such, the directional terminology is used for purposes of illustration and is in no way limiting. On the other hand, the drawings are only schematic and the sizes of components may be exaggerated for clarity. It is to be understood that other embodiments may be utilized and structural changes may be made without departing from the scope of the invention. Also, it is to be understood that the phraseology and terminology used herein are for the purpose of description and should not be regarded as limiting. The use of “including”, “comprising”, or “having” and variations thereof herein is meant to encompass the items listed thereafter and equivalents thereof as well as additional items. Unless limited otherwise, the terms “connected”, “coupled”, and “mounted” and variations thereof herein are used broadly and encompass direct and indirect connections, couplings, and mountings. Similarly, the terms “facing”, “faces”, and variations thereof herein are used broadly and encompass direct and indirect facing, and “adjacent to” and variations thereof herein are used broadly and encompass directly and indirectly “adjacent to”. Therefore, the description of “A” component facing “B” component herein may contain the situations that “A” component facing “B” component directly or one or more additional components is between “A” component and “B” component. Also, the description of “A” component “adjacent to” “B” component herein may contain the situations that “A” component is directly “adjacent to” “B” component or one or more additional components is between “A” component and “B” component. Accordingly, the drawings and descriptions will be regarded as illustrative in nature and not as restrictive.

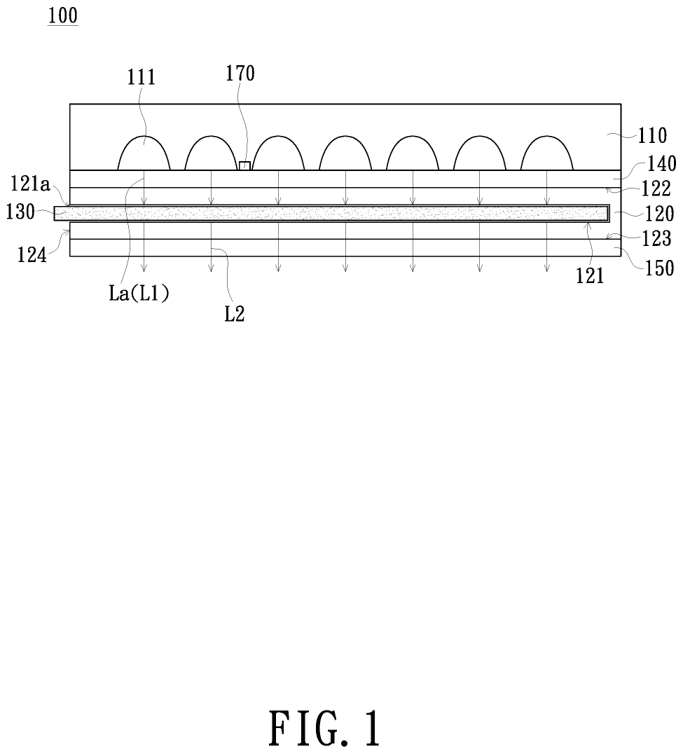

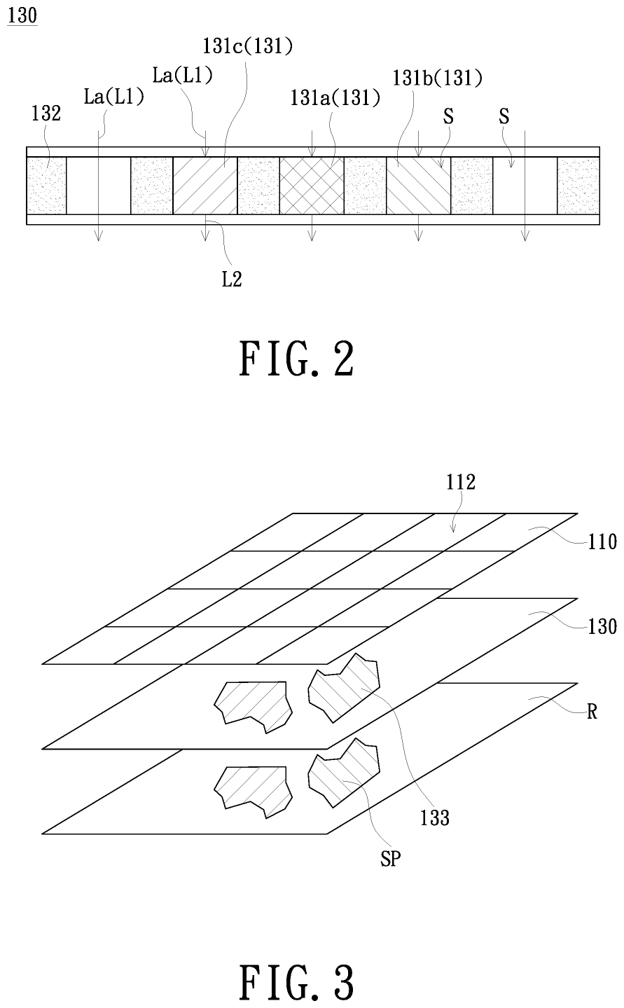

FIG. 1 is a schematic view of a phototherapy device according to an embodiment of the present invention. FIG. 2 is a schematic view of a light conversion device in FIG. 1. Referring to FIGS. 1 and 2, the phototherapy device 100 of this embodiment includes a light source module 110, a base 120 and at least one light conversion device 130. The base 120 has an installation slot 121. The light conversion devices 130 can be detachably arranged in the installation slot 121. In addition, each light conversion device 130 includes a plurality of light conversion patterns 131. The light source module 110 is arranged on a side of the base 120 and is configured to provide an excitation beam L1 to the light conversion pattern 131. The light conversion pattern 131 is configured to convert at least part of the excitation beam L1 into a converted beam L2 through excitation and other methods and then emit the converted beam L2. In FIG. 1, only one light conversion device 130 is shown for the illustration, but the phototherapy device 100 of this embodiment may include a plurality of light conversion devices 130. Because the light conversion device 130 can be detachably arranged in the installation slot 121, the user of the phototherapy device 100 can select and arrange a corresponding light conversion device 130 in the installation slot 121 according to different treatments or needs.

In this embodiment, the light source module 110 includes, for example, a plurality of light emitting elements 111 arranged in an array, but is not limited thereto. In other embodiments, the light source module 110 may include at least one light emitting element 111, and is not limited by the quantity and arrangement. The light emitting element 111 may be a light emitting diode (LED), but is not limited thereto. The light emitting element 111 is configured to provide a sub beam La, and the excitation beam L1 includes these sub beams La. The wavelength of the excitation beam L1 is, for example, 430 nm to 460 nm. The excitation beam L1 may have other suitable wavelength, depending on the corresponding light conversion device 130.

The base 120 has, for example, a light incident surface 122, a light emitting surface 123 and a side surface 124. The light incident surface 122 is opposite to the light emitting surface 123, and the side surface 124 is connected between the light incident surface 122 and the light emitting surface 123. The light source module 110 is arranged on the light incident surface 122. The excitation beam L1 from the light source module 110 is incident on the light conversion pattern 131 through the light incident surface 122, and the converted beam L2 converted by the light conversion pattern 131 exits through the light emitting surface 123. A slot opening 121a of the installation slot 121 is located on the side surface 124. The light conversion device 130 may be inserted into the installation slot 121 or drawn out from the installation slot 121 through the slot opening 121a.

In this embodiment, the phototherapy device 100 may further include a protective layer 140. The protective layer 140 is arranged between the base 120 and the light source module 110 and configured to avoid damage to the light source module 110 and the base 120. In addition, the material of the base 120 may be flexible or non flexible. The phototherapy device 100 can be designed as a contact phototherapy device or a non-contact phototherapy device in different embodiments. In the embodiment where the phototherapy device 100 is designed as a contact phototherapy device, for example, the base 120 is made of flexible material, the light source module 110 may be a flexible light source module, the light conversion device 130 may be a flexible light conversion device, and the protective layer 140 may be a flexible protective layer. In addition, in the embodiment where the phototherapy device 100 is a contact phototherapy device, the phototherapy device 100 may further include a skin friendly layer 150. The skin friendly layer 150 is arranged on the surface of the base 120 far from the light source module 110 (i.e., the light emitting surface 123) and configured to improve the use experience. The material of the skin friendly layer 150 has light transmission, which can be silica gel, but is not limited thereto.

The light conversion device 130 includes, for example, a plurality of light conversion patterns 131. The material of the light conversion pattern 131 is, for example, a color conversion material such as a quantum dot or phosphor, but is not limited thereto. The light conversion device 130 further includes, for example, a light shielding layer 132. The light shielding layer 132 is partitioned into a plurality of accommodating spaces S. The light conversion patterns 131 are respectively arranged in the accommodating spaces S.

The light emission wavelengths of the light conversion patterns 131 of the light conversion device 130 (i.e. the wavelengths of the converted beams L2) may be the same or different. Specifically, different wavelengths of light have different effects. For example, the red light has good penetration, which is helpful to activate blood circulation, relieve pain, and accelerate wound healing; the green light is helpful to relieve skin allergy and fatigue; the blue light is helpful to cure acne, prevent wound infection, sterilize, and inhibit oil secretion; and the infrared light is helpful to muscle repair and relieve joint pain. Therefore, each light conversion device 130 may include a variety of light conversion patterns 131 with different light emission wavelengths, such as the light conversion pattern 131a with a light emission wavelength of 500 nm to 540 nm (green light), the light conversion pattern 131b with a light emission wavelength of 610 nm to 650 nm (red light), and the light conversion pattern 131c with a light emission wavelength of 900 nm to 1250 nm (infrared light). In other embodiments, the light conversion pattern 131 of the light conversion device 130 may have only one light emission wavelength, two light emission wavelengths or more than three light emission wavelengths, depending on the design requirements.

In this embodiment, part of the accommodating spaces S are not provided with the light conversion pattern 131, so the excitation beam L1 can pass through the light conversion device 130 and irradiate on the user, and therefore phototherapy can be performed by the excitation beam L1 of the light source module 110. In one embodiment, the wavelength of the excitation beam L1 is, for example, 430 nm to 460 nm, thus the excitation beam L1 can provide the therapeutic effect of the aforementioned blue light. In addition, in the accommodating space S without the light conversion pattern 131, a through hole may be formed on the light conversion device 130 to allow the excitation beam L1 to pass therethrough directly, or a transparent material that allows the excitation beam L1 to pass therethrough is provided.

FIG. 3 is a schematic view of a light source module, a light conversion device and a light spot projected on a reference surface of a phototherapy device according to an embodiment of the present invention. In this embodiment, the structure of the phototherapy device 100 and the light conversion device 130 can refer to the foregoing figures, and no redundant detail is to be given herein. Please refer to FIGS. 1 to 3. In FIG. 3, the light conversion device 130 has, for example, two phototherapy patterns 133. The phototherapy pattern 133 is formed by an arrangement of a plurality of light conversion patterns 131 in FIG. 2. In addition, the light source module 110 may be designed to have a plurality of light emitting blocks 112. Each light emitting block 112 may include one or more light emitting elements 111 shown in FIG. 1. In this way, by controlling the light emitting block 112 corresponding to the phototherapy pattern 133 to emit light, the phototherapy pattern 133 can generate a light spot SP projected on the reference plane R. When the phototherapy device 100 performs phototherapy on the user, the reference plane R can be regarded as the user's skin surface. In another embodiment similar to the one of FIG. 3 in which there are two phototherapy patterns 133 on the light conversion device 130, each light emitting block 112 of the light source module 110 may be controlled to emit light, and the part of the light conversion device 130 outside the two phototherapy patterns 133 is not provided with a light conversion pattern 131 or is provided with a material for shielding light, such as light shielding or light absorption.

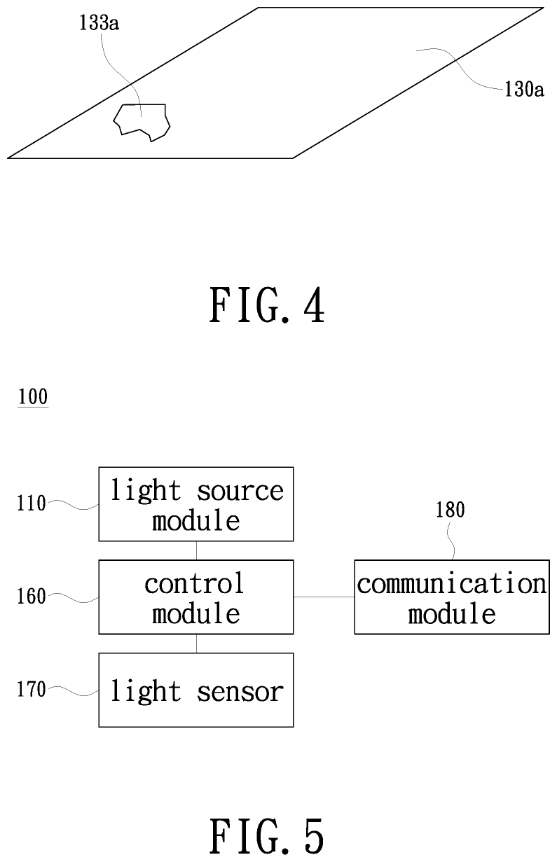

FIG. 4 is a schematic view of another light conversion device in the embodiment of FIG. 3. Referring to FIGS. 3 and 4, the quantity and shape of the phototherapy patterns 133a of the light conversion device 130a in FIG. 4 are different from those of the phototherapy patterns 133 of the light conversion device 130 in FIG. 3. In other words, the arrangement and distribution of light conversion patterns 131 (as shown in FIG. 2) of different light conversion devices (e.g., the light conversion devices 130, 130a) of the present invention can be different to form different phototherapy patterns 133, 133a. Therefore, the user can select a specific light conversion device (e.g., the light conversion device 130 or the light conversion device 130a) required for phototherapy according to different treatments or needs.

FIG. 5 is a block diagram of a phototherapy device according to an embodiment of the present invention. The structure of the light source module 110 and the light conversion device 130 can refer to the foregoing figures, and no redundant detail is to be given herein. Please refer to FIGS. 1, 3 and 5. The phototherapy device 100 of this embodiment may further include a control module 160. The control module 160 is electrically connected to the light emitting element 111 of the light source module 110 and configured to control the luminous intensity of the light emitting element 111. The luminous intensity referred to herein includes controlling the light emitting element 111 to emit light or not to emit light and the brightness when the light emitting element 111 emits light. In this way, the aforementioned control of the light emitting block 112 corresponding to the phototherapy pattern 133 to emit light is achieved.

Referring to FIGS. 1 and 5, the phototherapy device 100 may further include a light sensor 170. The light sensor 170 may be arranged on the same side of the base 120 as the light source module 110. Specifically, the light sensor 170 can be arranged between the light emitting elements 111 as shown in FIG. 1, or arranged beside the light source module 110. The light sensor 170 is, for example, electrically connected to the control module 160 and configured to sense the converted beam L2 reflected back by the user. In this way, the user's skin condition, such as the wound condition, can be analyzed by the optical signal received by the light sensor 170 for subsequent treatment.

In addition, the phototherapy device 100 may further include a communication module 180. The communication module 180 is electrically connected to the control module 160 and configured to transmit information between the phototherapy device 100 and an external device (e.g., a host of a medical unit). The information transmission is, for example, receiving a control signal from the external device, transmitting the optical signal received by the light sensor 170 to the external device, and transmitting the use time of the phototherapy device 100 or other use information to the external device.

In summary, in the phototherapy device of each embodiment of the present invention, because the base has an installation slot allowing the light conversion device to have a detachable arrangement, the user can select or replace a specific light conversion device to convert the excitation beam emitted by the light source module into a beam with a specific wavelength and phototherapy pattern required for phototherapy according to different treatments or needs in a single phototherapy device.

The foregoing description of the preferred embodiment of the invention has been presented for purposes of illustration and description. It is not intended to be exhaustive or to limit the invention to the precise form or to exemplary embodiments disclosed. Accordingly, the foregoing description should be regarded as illustrative rather than restrictive. Obviously, many modifications and variations will be apparent to practitioners skilled in this art. The embodiments are chosen and described in order to best explain the principles of the invention and its best mode practical application, thereby to enable persons skilled in the art to understand the invention for various embodiments and with various modifications as are suited to the particular use or implementation contemplated. It is intended that the scope of the invention be defined by the claims appended hereto and their equivalents in which all terms are meant in their broadest reasonable sense unless otherwise indicated. Therefore, the term “the invention”, “The invention” or the like is not necessary limited the claim scope to a specific embodiment, and the reference to particularly preferred exemplary embodiments of the invention does not imply a limitation on the invention, and no such limitation is to be inferred. The invention is limited only by the spirit and scope of the appended claims. Moreover, these claims may refer to use “first”, “second”, etc. following with noun or element. Such terms should be understood as a nomenclature and should not be construed as giving the limitation on the number of the elements modified by such nomenclature unless specific number has been given. The abstract of the disclosure is provided to comply with the rules requiring an abstract, which will allow a searcher to quickly ascertain the subject matter of the technical disclosure of any patent issued from this disclosure. It is submitted with the understanding that it will not be used to interpret or limit the scope or meaning of the claims. Any advantages and benefits described may not apply to all embodiments of the invention. It should be appreciated that variations may be made in the embodiments described by persons skilled in the art without departing from the scope of the invention as defined by the following claims. Moreover, no element and component in the disclosure is intended to be dedicated to the public regardless of whether the element or component is explicitly recited in the following claims.

What is claimed is:

1. A phototherapy device, comprising:
a base, having an installation slot;
at least one light conversion device, detachably arranged in the installation slot, wherein each of the at least one light conversion device comprises a plurality of light conversion patterns; and
a light source module, arranged on a side of the base and configured to provide an excitation beam to the light conversion patterns, so that each of the light conversion patterns emits a converted beam.

2. The phototherapy device according to claim 1, wherein the base has a light incident surface, a light emitting surface and a side surface, the light incident surface is opposite to the light emitting surface, the side surface is connected between the light incident surface and the light emitting surface, the light source module is arranged on the light incident surface, the converted beam exits through the light emitting surface, and a slot opening of the installation slot is located on the side surface.

3. The phototherapy device according to claim 1, wherein the light conversion patterns comprise a variety of light conversion patterns with different light emission wavelengths.

4. The phototherapy device according to claim 1, wherein each of the at least one light conversion device further comprises a light shielding layer, the light shielding layer is partitioned into a plurality of accommodating spaces, and the light conversion patterns are respectively arranged in the accommodating spaces.

5. The phototherapy device according to claim 4, wherein part of the accommodating spaces is not provided with the light conversion pattern.

6. The phototherapy device according to claim 1, wherein a quantity of the at least one light conversion device is plural, an arrangement and distribution of the light conversion patterns of the light conversion devices are different, and one of the light conversion device is selected to arrange in the installation slot.

7. The phototherapy device according to claim 1, further comprising a control module, wherein the light source module comprises a plurality of light emitting elements arranged in an array, and the control module is electrically connected to the light emitting elements and configured to control a luminous intensity of the light emitting elements.

8. The phototherapy device according to claim 7, further comprising a communication module, electrically connected to the control module.

9. The phototherapy device according to claim 7, further comprising a light sensor, arranged on the side of the base and configured to sense the converted beam reflected back by a user.

10. The phototherapy device according to claim 1, further comprising a protective layer, arranged between the base and the light source module.

11. The phototherapy device according to claim 1, further comprising a skin friendly layer, arranged on a surface of the base far from the light source module.
